(19) United States
(12) Patent Application Publication
(10) Pub. No.: US 2021/0161965 A1
(43) Pub. Date: Jun. 3, 2021

(54) KIR3DL1/HLA-B SUBTYPES FOR HEMATOPOIETIC CELL TRANSPLANTATION DONOR SELECTION

(71) Applicant: MEMORIAL SLOAN KETTERING CANCER CENTER, New York, NY (US)

(72) Inventor: Katharine Hsu, New York, NY (US)

(73) Assignee: MEMORIAL SLOAN KETTERING CANCER CENTER, New York, NY (US)

(21) Appl. No.: 16/493,264

(22) PCT Filed: Mar. 9, 2018
(86) PCT No.: PCT/US2018/021819

§ 371 Date: Sep. 11, 2019

Related U.S. Application Data
(60) Provisional application No. 62/470,272, filed on Mar. 12, 2017.

(51) Int. Cl. A61K 35/28; C12Q 1/6881; A61K 35/02

(52) U.S. Cl. CPC A61K 35/28; C12Q 1/6881; C12Q 2600/118; C12Q 2600/156; C12Q 2600/158; A61K 35/02

(57) ABSTRACT

The present disclosure relates generally to methods for treating leukemia in a subject in need thereof comprising administering to the subject an effective amount of hematopoietic cells obtained from a candidate donor having a specific combination of KIR3DL1 and HLA-B alleles. Also disclosed herein are methods for protecting a leukemia patient recipient from leukemic relapse following allogeneic hematopoietic cell transplantation (HCT).

### CROSS-REFERENCE TO RELATED APPLICATIONS

This application claims the benefit of and priority to U.S. Provisional Appl. No. 62/470,272, filed Mar. 12, 2017, the disclosure of which is incorporated by reference herein in its entirety.

### GOVERNMENT SUPPORT

This invention was made with government support under HL088134, AI069197 and CA023766 awarded by the National Institutes of Health. The government has certain rights in the invention.

### TECHNICAL FIELD

The present technology relates generally to methods for treating leukemia in a subject in need thereof comprising administering to the subject an effective amount of hematopoietic cells obtained from a candidate donor having a specific combination of KIR3DL1 and HLA-B alleles. Also disclosed herein are methods for protecting a leukemia patient recipient from leukemic relapse following allogeneic hematopoietic cell transplantation (HCT).

### BACKGROUND

The following description of the background of the present technology is provided simply as an aid in understanding the present technology and is not admitted to describe or constitute prior art to the present technology.

As the only known curative therapy for most persons with acute myelogenous leukemia (AML), allogeneic hematopoietic cell transplantation (HCT) enlists an immune-mediated graft-vs-leukemia alloreactivity distinct from graft-vs-host disease (GvHD) (Duval M, et al., Journal of Clinical Oncology 28:3730-3738 (2010)). Although GvHD is significantly reduced with greater HLA-matching, relapse still occurs in 37% of patients (MC P, Zhu X, CIBMTR Summary Slides (2015)), suggesting that immune-mediated AML control differs between donors. For patients undergoing allogeneic HCT, donor preference is given to those matched for up to 12 HLA alleles. While GvHD and graft rejection have decreased due to tighter HLA matching, risks of leukemic relapse and overall mortality remain disappointingly high.

### SUMMARY OF THE PRESENT TECHNOLOGY

In one aspect, the present disclosure provides a method for treating leukemia in a subject in need thereof, comprising administering to the subject an effective amount of hematopoietic cells obtained from a candidate donor, wherein the candidate donor has a KIR3DL1-HLA-B allele combination of KIR3DL1-N and HLA-Bw4. In some embodiments, the HLA-Bw4 allele is HLA-Bw4-80T or HLA-Bw4-80I. Additionally or alternatively, in some embodiments, the candidate donor expresses KIR2DS1 and HLA-C1. In certain embodiments, the candidate donor expresses HLA-C1 or HLA-C2. In some embodiments, the subject is human.

The leukemia may be a chronic leukemia or acute leukemia. In some embodiments, the leukemia is acute myelogenous leukemia (AML), chronic myelogenous leukemia (CIVIL), chronic lymphocytic leukemia (CLL), acute lymphocytic leukemia (ALL), hairy cell leukemia, T cell prolymphcytic leukemia (T-PLL), or large granular lymphocytic leukemia.

Additionally or alternatively, in some embodiments, the hematopoietic cells comprise hematopoietic stem cells. In certain embodiments, the hematopoietic cells obtained from the candidate donor are derived from bone marrow, peripheral blood cells, or umbilical cord.

Additionally or alternatively, in some embodiments, the method further comprises treating the subject with chemotherapy and/or radiation.

In another aspect, the present disclosure provides a method for protecting a leukemia patient from leukemic relapse following allogeneic hematopoietic cell transplantation (HCT), comprising administering to the leukemia patient an allogeneic hematopoietic graft derived from a candidate donor, wherein the candidate donor has a KIR3DL1-HLA-B allele combination of KIR3DL1-N and HLA-Bw4. In some embodiments, the HLA-Bw4 allele is HLA-Bw4-80T or HLA-Bw4-80I. Additionally or alternatively, in some embodiments, the candidate donor expresses KIR2DS1 and HLA-C1. In certain embodiments, the candidate donor expresses HLA-C1 or HLA-C2.

The leukemia may be a chronic leukemia or acute leukemia. In some embodiments, the leukemia is acute myelogenous leukemia (AML), chronic myelogenous leukemia (CIVIL), chronic lymphocytic leukemia (CLL), acute lymphocytic leukemia (ALL), hairy cell leukemia, T cell prolymphcytic leukemia (T-PLL), or large granular lymphocytic leukemia. In some embodiments, the leukemia patient is at the early, intermediate or advanced stage of leukemia.

Additionally or alternatively, in some embodiments, the allogeneic hematopoietic graft is a bone marrow graft or a peripheral bone stem cell graft. In certain embodiments of the method, the hematopoietic cell transplantation is ablative, T-cell depleted, or T-cell replete. Additionally or alternatively, in some embodiments, the leukemia patient does not suffer from GvHD after the allogeneic hematopoietic cell transplantation.

### BRIEF DESCRIPTION OF THE DRAWINGS

FIG. 1(A) shows primary KIR3DL1-low or -high NK cells from healthy Bw4-80T⁺ or Bw4-80I⁺ donors challenged with 721.221 cells transfected with HLA-B*44 or HLA-B*51, respectively. % inhibition of KIR3DL1⁺ NK cells was calculated by comparing degranulation of the same NK cells toward parental 721.221 or 721.221-Bw4⁺ cells. Each bar represents 10-15 healthy donors.

FIG. 1(B) demonstrates degranulation of KIR3DL1-low (l) or KIR3DL/-high (h)+ NK cells derived from Bw4-80T⁺ donors in response to challenge with the Bw4-80T⁺ AML cell line, SET-2. % inhibition was calculated by comparing NK degranulation in the presence and absence of the KIR3DL1 blocking antibody, DX9.

FIG. 1(C) shows demonstrates degranulation of KIR3DL1-low (l) or KIR3DL1-high (h) NK cells derived from Bw4-80I⁺ donors in response to challenge with the Bw4-80I+ AML cell line, KG-1.

FIG. 1(D) shows the response of KIR3DL1-null or inhibitory KIR-negative cells to HLA-negative 721.221 target cells. NK cells are segregated based on the presence or absence of HLA-Bw4 in the donor.

FIG. 1(E) shows KIR3DL1-null expressing NK cells from Bw4-80T⁺ or Bw4-80I⁺ donors challenged using 721.221 cells transfected for expression of HLA-B*44 or HLA-B*51, respectively. % inhibition is calculated by comparing responsiveness against parental 721.221 cells and Bw4-transfected cells. Each bar represents 4-6 donors and the mean +/−SEM.

FIG. 2(A) demonstrates the cytotoxicity of the Bw4-80T⁺ AML cell line SET-2 by PBMC from Bw4-80T⁺ donors. All bars represent means +/−SEM. Each bar represents a minimum of 6 independent healthy donors and HLA-C subtype groups are stratified equivalently between groups.

FIG. 2(B) demonstrates the cytotoxicity of the Bw4-80I⁺ AML cell line KG-1 by PBMC from Bw4-80I⁺ donors. All bars represent means +/−SEM. Each bar represents a minimum of 6 independent healthy donors and HLA-C subtype groups are stratified equivalently between groups.

FIG. 2(C) demonstrates the cytotoxicity of the Bw4-80T⁺ AML cell line SET-2 by PBMC from Bw4-80T⁺ donors in the presence of Z27 antibody. All bars represent means +/−SEM. Each bar represents a minimum of 6 independent healthy donors and HLA-C subtype groups are stratified equivalently between groups.

FIG. 2(D) demonstrates the cytotoxicity of the Bw4-80I⁺ AML cell line KG-1 by PBMC from Bw4-80I⁺ donors in the presence of Z27 antibody. All bars represent means +/−SEM. Each bar represents a minimum of 6 independent healthy donors and HLA-C subtype groups are stratified equivalently between groups.

FIG. 3(A) shows cumulative incidence curves for relapse among 1328 patients with AML who underwent HCT. The indicated hazard ratios and p-values compare strongly-interacting pairs with weak and non-inhibition pairs combined and reflect adjustment for patient's age, conditioning regimen, T-cell depletion, graft type, disease status, CMV match and gender match. All curve comparisons were completed using Cox regression analysis for the time-to-event post-HCT outcomes.

FIG. 3(B) shows a Kaplan-Meier plot for survival among all donor-patient pairs among 1328 patients with AML who underwent HCT. The indicated hazard ratios and p-values compare strongly-interacting pairs with weak and non-inhibition pairs combined and reflect adjustment for patient's age, conditioning regimen, T-cell depletion, graft type, disease status, CMV match and gender match. All curve comparisons were completed using Cox regression analysis for the time-to-event post-HCT outcomes.

FIG. 3(C) shows cumulative incidence curves for relapse among 606 patients with AML that exhibited HLA-C1 and HLA-C2 who underwent HLA-matched HCT. The indicated hazard ratios and p-values compare strongly-interacting pairs with weak and non-inhibition pairs combined and reflect adjustment for patient's age, conditioning regimen, T-cell depletion, graft type, disease status, CMV match and gender match. All curve comparisons were completed using Cox regression analysis for the time-to-event post-HCT outcomes.

FIG. 3(D) shows a Kaplan-Meier plot for survival among 606 patients with AML that exhibited HLA-C1 and HLA-C2 who underwent HLA-matched HCT. The indicated hazard ratios and p-values compare strongly-interacting pairs with weak and non-inhibition pairs combined and reflect adjustment for patient's age, conditioning regimen, T-cell depletion, graft type, disease status, CMV match and gender match. All curve comparisons were completed using Cox regression analysis for the time-to-event post-HCT outcomes.

FIG. 4 shows cumulative incidence curves for relapse among 1328 patients with AML who underwent HCT from an unrelated donor. Patients with beneficial KIR2DS1+HLA-C1 are shown as solid lines; patients lacking KIR2DS1 and/or HLA-C1 are shown in dashed lines. Patients with strong inhibiting partnerships of KIR3DL1 and HLA-B are shown as black lines; patients with weak inhibiting/non-inhibiting partnerships of KIR3DL1 and HLA-B are shown as red lines. Hazard ratios compare the indicated groups and patients with neither KIR2DS1/KIR3DL1 benefit to those with both.

FIG. 5(A) shows overall relapse among HCT pairs with specific donor KIR3DL1 and HLA-B subtype combinations. Relative hazards were calculated by Cox regression analysis to compare the impacts of donor and recipient HLA-B. Diamonds (⋄) represent KIR3DL1-N donors, triangles (Δ) represent KIR3DL1-L donors and circles (◯) represent KIR3DL1-H donors. Open, grey and black symbols represent donor/recipients encoding Bw6, Bw4-80T or Bw4-80I, respectively, and the numbers of donor-patient pairs in each compound subgroup are shown. Relative hazards reflect adjustment for patient's age, conditioning regimen, T-cell depletion, graft type, disease status, CMV and gender match. The legend indicates the number of patients present in each subgroup assessed.

FIG. 5(B) shows mortality among HCT pairs with specific donor KIR3DL1 and HLA-B subtype combinations. Relative hazards were calculated by Cox regression analysis to compare the impacts of donor and recipient HLA-B. Diamonds (⋄) represent KIR3DL1-N donors, triangles (Δ) represent KIR3DL1-L donors and circles (◯) represent KIR3DL1-H donors. Open, grey and black symbols represent donor/recipients encoding Bw6, Bw4-80T or Bw4-80I, respectively, and the numbers of donor-patient pairs in each compound subgroup are shown. Relative hazards reflect adjustment for patient's age, conditioning regimen, T-cell depletion, graft type, disease status, CMV and gender match. The legend indicates the number of patients present in each subgroup assessed.

FIG. 5(C) shows relapse segregated by recipient HLA-B subtype (Bw6, Bw4-80T or Bw4-80I) stratified by donor KIR3DL1 subtypes (KIR3DL1-N, -L or -H). Relative hazards were calculated by Cox regression analysis to compare the impacts of donor and recipient HLA-B. Diamonds (⋄) represent KIR3DL1-N donors, triangles (Δ) represent KIR3DL1-L donors and circles (◯) represent KIR3DL1-H donors. Open, grey and black symbols represent donor/recipients encoding Bw6, Bw4-80T or Bw4-80I, respectively, and the numbers of donor-patient pairs in each compound subgroup are shown. Relative hazards reflect adjustment for patient's age, conditioning regimen, T-cell depletion, graft type, disease status, CMV and gender match. The legend indicates the number of patients present in each subgroup assessed.

FIG. 5(D) shows mortality segregated by recipient HLA-B subtype (Bw6, Bw4-80T or Bw4-80I) stratified by donor KIR3DL1 subtypes (KIR3DL1-N, -L or -H). Relative hazards were calculated by Cox regression analysis to compare the impacts of donor and recipient HLA-B. Diamonds (⋄) represent KIR3DL1-N donors, triangles (Δ) represent KIR3DL1-L donors and circles (◯) represent KIR3DL1-H donors. Open, grey and black symbols represent donor/recipients encoding Bw6, Bw4-80T or Bw4-80I, respectively, and the numbers of donor-patient pairs in each compound subgroup are shown. Relative hazards reflect adjustment for patient's age, conditioning regimen, T-cell depletion, graft type, disease status, CMV and gender match. The legend indicates the number of patients present in each subgroup assessed.

FIG. 6(A) shows total HLA class I expression at rest and following stimulation with IFN-γ in PBMCs from health donors or primary AML blasts from 12 patients that were stained for CD33. Three patients with AML and three HLA-B matched healthy donors matched for HLA-B subtypes are shown and are representative of 3-4 samples per HLA-B subtype analyzed. Values indicate the mean fluorescence intensities of HLA-ABC staining among CD33⁺ and CD33⁻ populations.

FIG. 6(B) shows Bw4 expression at rest and following stimulation with IFN-γ in PBMCs from health donors or primary AML blasts from 12 patients that were stained for CD33. Three patients with AML and three HLA-B matched healthy donors matched for HLA-B subtypes are shown and are representative of 3-4 samples per HLA-B subtype analyzed. Values indicate the mean fluorescence intensities of Bw4 staining among CD33⁺ and CD33⁻ populations.

FIG. 6(C) shows nine cell lines of differing HLA-B epitope backgrounds are stained for total HLA class I expression and Bw4 at rest (grey solid histograms) and following stimulation with IFN-γ (black dashed histograms). Control (unstained) cells are shown as filled light grey histograms. The B lymphoid cell line, 721.221, which does not express HLA, is shown for comparison. The cell lines, ML-2, OC-1, MO-91 and Kasumi-1 exhibit HLA-A epitopes that contain Bw4 motifs. Data represent two independent trials and numbers indicate the mean fluorescent intensities.

FIG. 7(A) shows the assessment of intracellular KIR3DL1-n. Cells were permeabilized and stained with anti-KIR3DL1 clone 177407. Staining was optimized on NK cells from KIR3DL1-n homozygous donors.

FIG. 7(B) shows the assessment of intracellular KIR3DL1-n. Cells were permeabilized and stained with anti-KIR3DL1 clone 177407. Staining was verified on NK cells from donors exhibiting KIR3DL1-n+KIR3DL1-h.

FIG. 7(C) shows the assessment of intracellular KIR3DL1-n. Cells were permeabilized and stained with anti-KIR3DL1 clone 177407. Staining was verified on NK cells from KIR3DL1-h homozygous donors.

FIG. 8(A) demonstrates the cytotoxicity of the Bw4-80T+ AML cell line SET-2 by PBMC from Bw4-80I⁺ healthy donors co-expressing KIR3DL1-l and KIR3DL1-h (l+h) or exhibiting only one of KIR3DL1-h or KIR3DL1-l. Bars represent means +/−SEM and a minimum of 7 independent donors and three independent trials. Means are compared by one-way ANOVA using Tukey's post-hoc test. *, p<0.05; **, p<0.01.

FIG. 8(B) demonstrates the cytotoxicity of the Bw4-80I+ AML cell line KG-1 by PBMC from healthy Bw4-80I+ donors co-expressing KIR3DL1-l and KIR3DL1-h (l+h) or exhibiting only one of KIR3DL1-h (H) or KIR3DL1-l (L). Bars represent means +/−SEM and a minimum of 7 independent donors and three independent trials. Means are compared by one-way ANOVA using Tukey's post-hoc test. *, p<0.05; **, p<0.01.

FIG. 9 shows the impact of KIR3DL1/HLA-B subtype combinations. In addition to the indicated adjustments (KIR2DS1, Cen-BB), all models were adjusted for donor age, treatment regimen, T-cell depletion, graft type, disease status, HLA match, CMV and gender match. ^{A}Donors with KIR3DL1-L+Bw4-80T or KIR3DL1-H+Bw4-80I. ^{B}Donors with any KIR3DL1+Bw6/Bw6, or with KIR3DL1-N+Bw4-80I or KIR3DL1-N+Bw4-80T. ^{C}Donors with KIR3DL1-H+Bw4-80T or KIR3DL1-L+Bw4-80I. ^{D}KIR2DS1 effect was defined by donors exhibiting KIR2DS1 and HLA-C1 vs all others. ^{E}Cen-BB in donors was defined as KIR2DL2⁺ and/or KIR2DS2⁺ and KIR2DL3-negative.

FIG. 10 shows donor, recipient and transplant characteristics according to disease and KIR3DL1/HLA-B subtypes. ^{α}Low risk indicates first complete remission, intermediate risk second or higher complete remission, and high risk primary induction failure or relapse. ^{β}Race and ethnic groups were self-reported. ^{γ}HLA donor-recipient matches at HLA A, B, C, DRB1, DQB1.

FIG. 11 shows the donor KIR3DL1 and HLA-B subtype distributions.

FIG. 12 shows alleles comprising KIR3DL1 subtype groups and their differential primer binding sites. The polymorphic sites that differentiate allele subtypes are shown in bold and column labels indicate the polymorphic site in the mature coding sequence. ^{#}KIR3DS1 alleles are differentiated from KIR3DL1*002-group high alleles by product size; intron 3 in KIR3DS1 alleles is 200 bp longer than that of KIR3DL1. Banded rows indicate alleles identified by medium resolution sequence-specific primed PCR (PCR-SSP).

FIG. 13 shows the antibody clones and sources used for flow cytometry.

FIG. 14 shows combined benefits mediated by KIR3DL1 and KIR2DS1.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

The present disclosure demonstrates that donors possessing KIR3DL1-N and HLA-Bw4 allele combinations exhibit low inhibitory interactions, and that hematopoietic cell transplants from such donors were associated with lower relapse and higher survival, and, were not associated with a higher risk for GvHD. Nearly all patients exhibited the same HLA-B epitopes (Bw6, Bw4-80T, Bw4-80I) as their donors, indicating that even in HLA-matched allogeneic HCT, epistatic interactions between donor KIR and HLA class I at the allotype resolution can have a profound impact on transplant outcome. The findings disclosed herein confirm the importance of the interaction between KIR3DL1 and HLA-B allotypes in determining HCT outcomes. The present disclosure demonstrates that KIR3DL1 allele typing to select stem cell donors with favorable KIR3DL1-HLA-B allele combinations minimizes inhibition potential, maximizes leukemic toxicity, lowers relapse rates and increases patient survival.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of AML. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, the terms “individual”, “patient”, or “subject” can be an individual organism, a vertebrate, a mammal, or a human. In some embodiments, the individual, patient or subject is a human.

A “KIR3DL1-h” allele, as used herein, refers to an allele which expresses the KIR3DL1 receptor at high densities on the cell surface of NK cells detectable by cell surface staining, or an allele which is yet to be characterized for surface staining but shares substantial sequence similarity to an allele which expresses the KIR3DL1 receptor at high densities on the cell surface of NK cells detectable by cell surface staining. Cell surface staining can be performed using an antibody directed to KIR3DL1 receptor. Examples of suitable antibodies include Z27 or DX9, both widely available, for example, from BD Biosciences (San Jose, Calif.) or Thermo Fisher Scientific (Waltham, Mass.).

A “KIR3DL1-l” allele, as used herein, refers to an allele which expresses the KIR3DL1 receptor at low densities on the cell surface of NK cells detectable by cell surface staining (e.g., using Z27 or DX9), or an allele which is yet to be characterized for surface staining but shares substantial sequence similarity to an allele which expresses the KIR3DL1 receptor at low densities on the cell surface of NK cells detectable by cell surface staining.

A “KIR3DL1-n” allele, as used herein, refers to an allele which expresses KIR3DL1 molecules retained intracellularly and not detectable by cell surface staining (e.g., using Z27 or DX9).

As used herein, “prevention” or “preventing” of a disorder or condition refers to a compound that, in a statistical sample, reduces the occurrence of the disorder or condition in the treated sample relative to an untreated control sample, or delays the onset of one or more symptoms of the disorder or condition relative to the untreated control sample. As used herein, preventing AML, includes preventing or delaying the initiation of symptoms of AML. As used herein, prevention of AML also includes preventing a recurrence of one or more signs or symptoms of AML.

As used herein, the term “sample” means biological sample material derived from living cells of a subject. Biological samples may include tissues, cells, protein or membrane extracts of cells, and biological fluids (e.g., ascites fluid or cerebrospinal fluid (CSF)) isolated from a subject, as well as tissues, cells and fluids (blood, plasma, saliva, urine, serum etc.) present within a subject.

As used herein, “substantial sequence similarity”, means that the relevant sequences share at least about 90%, 95%, 98%, 99% or higher identity at the nucleotide level, or at least about 90%, 95%, 98%, 99% or higher similarity or identity at the amino acid level.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Natural Killer Receptors

Natural killer (NK) cells are innate lymphocytes capable of recognizing transformed cells. Killer immunoglobulin-like receptors (KIR) control NK function and are encoded by the highly polymorphic, multi-membered KIR gene family (Parham P., Nat Rev Immunol 5:201-214 (2005)). Interaction between self-specific inhibitory KIR and cognate HLA ligands is fundamental to NK “education” (Kim S et al., Nature 436:709-713 (2005)), where cells expressing inhibitory KIR for self-HLA are “licensed” and more responsive than their “unlicensed” counterparts (Kim S et al., Nature 436:709-713 (2005); Anfossi N et al., Immunity 25:331-342 (2006)). Inflammatory cytokines induced following HCT (Niederwieser D et al., Transplantation 50:620-625 (1990)), activate unlicensed NK cells, but concurrently prompt HLA upregulation on the tumour, putting the educated NK population at risk for inhibition.

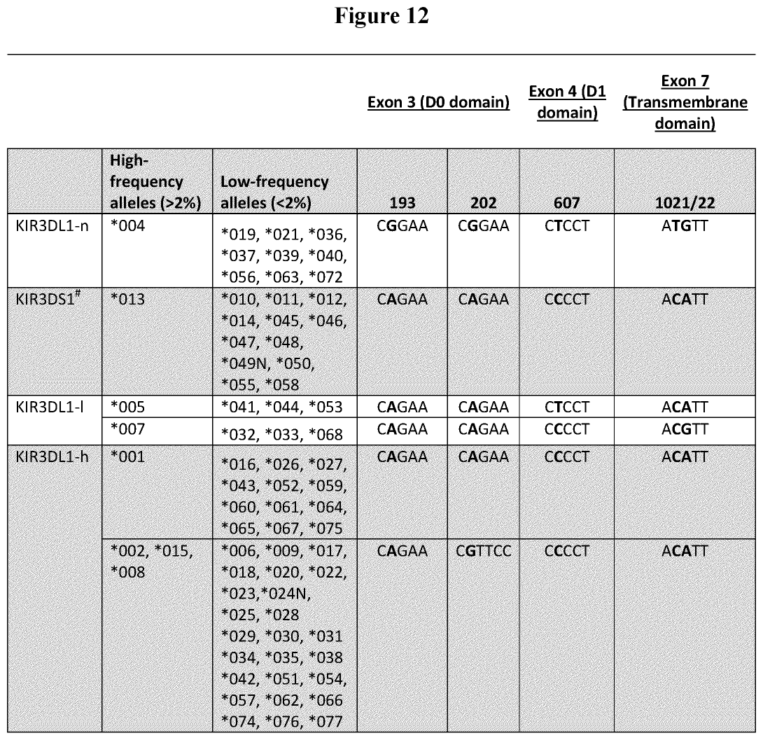

KIR3DL1-h alleles are those alleles which have been characterized by cell surface staining, including but not limited to KIR3DL1* 001, *002, *008, *015, *020, *033, and *052. Alleles yet to be characterized for surface staining but which share substantial sequence similarity to a KIR3DL1-h allele characterized by high density cell surface staining include but are not limited to *009, *016, *043, *067, *026, *052, *034, *035, *022, *017, *066, *029, *038, *025, *054, *018, *051, *023, *028, *062, *030, *024N, *031, *059, *060, *061, *064, *065, *074, *075, *076, *077, and *057. See FIG. 12.

KIR3DL1-l alleles which have been characterized by cell surface staining include but are not limited to KIR3DL1*005, *007, and *053. Alleles yet to be characterized for surface staining but which share substantial sequence similarity to a KIR3DL1-l allele characterized by low density cell surface staining include but not limited to *032, *033, *068, *044, and *041. See FIG. 12.

KIR3DL1-n alleles include but are not limited to *004, *019, and *056. Alleles yet to be characterized for surface staining but which share substantial sequence similarity to a KIR3DL1-n allele characterized by low density cell surface staining include but not limited to *021, *036, *037, *039, *056, *072, *063, and *040. See FIG. 12.

A KIR3DS1 allele expresses KIR3DS1 molecules, detectable by surface staining with Z27 but not DX9. KIR3DS1 alleles include but are not limited to KIR3DS1 *013, *047, *010, *011, *012, *014, *045, *046, *048, *049N, *050, *055, and *058. See FIG. 12.

A genomic DNA-containing sample may be obtained from a candidate donor matched for HLA-Bw4. The sample can be a tissue or blood sample, including, but not limited to, blood, fractions of blood, peripheral blood cells, skin or tissue biopsies, buccal swab samples, and umbilical cord blood. In some embodiments, the sample is processed to permit allele typing, e.g., a cell containing fraction is obtained from the sample, and genomic DNA is isolated. In other embodiments, a sample is used directly in allele typing.

The genomic DNA from the sample is then analyzed to determine whether the KIR3DL1 gene is present (96% of individuals), and which allele(s), KIR3DL1-h, KIR3DL1-l, KIR3DL1-n, or KIR-3DS1, is present; i.e., allele typing of the KIR3DL1 gene. Allele typing of the KIR3DL1 gene is achieved by using various approaches described in the art, including, but not limited to, hybridization based on sequence-specific oligonucleotides, sequencing, PCR-SSP (“sequence-specific primer”), and combinations thereof. FIG. 12 describes differential primer binding sites for each of the KIR3DL1 subtype groups.

Once the KIR3DL1 allele typing information is obtained, a donor can be assigned to one of the following subtype groups based on its allele combination: KIR3DL1-H (KIR3DL1 *h/*h, or KIR3DL1 *h/KIR3DS1), KIR3DL1-L (KIR3DL1 *l/*l, KIR3DL1 *l/*h, or KIR3DL1 *l/KIR3DS1), or KIR3DL1-N (KIR3DL1 *n/*n, KIR3DL1 *n/*h, KIR3DL1 *n/*l, or KIR3DL1 *n/KIR3DS1). One can then determine the inhibition potential of the donor KIR3DL1 subtype and the HLA-Bw4 allele combination. A donor can then be selected on the basis that the donor and recipient genotypes provide a low inhibitory combination of donor KIR3DL1 and donor/recipient HLA-Bw4 alleles. Such donor is associated with a reduced risk of leukemic relapse and increased rate of survival in the patient recipient.

### Treatment Methods of the Present Technology

As disclosed herein, the combination of KIR3DL1-N and HLA-Bw4 shows the lowest inhibitory potential. This finding was unexpected given that prior studies have suggested that KIR3DL1-n donors should be avoided (Niederwieser D et al., Transplantation 50:620-625 (1990); Brouwer R E et al., HIM 63:200-210 (2002)).

KIR3DL1-L and HLA-Bw4-80I, and most KIR3DL1-H and HLA-Bw4-80T combinations represent low inhibitory combinations, whereas KIR3DL1-H and HLA-Bw4-80I, and KIR3DL1-L and HLA-Bw4-80T generally represent high inhibitory combinations. HLA-B*2705 is a Bw4-80T allele but appears to have a highly inhibitory relationship with KIR3DL1-H. For purposes of this determination, donors are considered Bw4-80I, Bw4-80T, or Bw4-negative (Bw6/Bw6). All donors positive for Bw4-80I, homozygous or heterozygous, are assigned to the Bw4-80I group, independent of the presence/absence of HLA-Bw4-80T. Donors that are Bw4-80T/Bw4-80T or Bw6/Bw4-80T are classified to the Bw4-80T group.

In one aspect, the present disclosure provides a method for treating leukemia in a subject in need thereof, comprising administering to the subject an effective amount of hematopoietic cells obtained from a candidate donor, wherein the candidate donor has a KIR3DL1-HLA-B allele combination of KIR3DL1-N and HLA-Bw4. In some embodiments, the HLA-Bw4 allele is HLA-Bw4-80T or HLA-Bw4-80I. Additionally or alternatively, in some embodiments, the candidate donor expresses KIR2DS1 and HLA-C1. In certain embodiments, the candidate donor expresses HLA-C1 or HLA-C2. In some embodiments, the subject is human.

The leukemia may be a chronic leukemia or acute leukemia. In some embodiments, the leukemia is acute myelogenous leukemia (AML), chronic myelogenous leukemia (CIVIL), chronic lymphocytic leukemia (CLL), acute lymphocytic leukemia (ALL), hairy cell leukemia, T cell prolymphcytic leukemia (T-PLL), or large granular lymphocytic leukemia.

Additionally or alternatively, in some embodiments, the hematopoietic cells comprise hematopoietic stem cells. In certain embodiments, the hematopoietic cells obtained from the candidate donor are derived from bone marrow, peripheral blood cells, or umbilical cord.

Additionally or alternatively, in some embodiments, the method further comprises treating the subject with chemotherapy and/or radiation.

In another aspect, the present disclosure provides a method for protecting a leukemia patient from leukemic relapse following allogeneic hematopoietic cell transplantation (HCT), comprising administering to the leukemia patient an allogeneic hematopoietic graft derived from a candidate donor, wherein the candidate donor has a KIR3DL1-HLA-B allele combination of KIR3DL1-N and HLA-Bw4. In some embodiments, the HLA-Bw4 allele is HLA-Bw4-80T or HLA-Bw4-80I. Additionally or alternatively, in some embodiments, the candidate donor expresses KIR2DS1 and HLA-C1. In certain embodiments, the candidate donor expresses HLA-C1 or HLA-C2.

The leukemia may be a chronic leukemia or acute leukemia. In some embodiments, the leukemia is acute myelogenous leukemia (AML), chronic myelogenous leukemia (CIVIL), chronic lymphocytic leukemia (CLL), acute lymphocytic leukemia (ALL), hairy cell leukemia, T cell prolymphcytic leukemia (T-PLL), or large granular lymphocytic leukemia. In some embodiments, the leukemia patient is at the early, intermediate or advanced stage of leukemia.

Additionally or alternatively, in some embodiments, the allogeneic hematopoietic graft is a bone marrow graft or a peripheral bone stem cell graft. In certain embodiments of the method, the hematopoietic cell transplantation is ablative, T-cell depleted, or T-cell replete. Additionally or alternatively, in some embodiments, the leukemia patient does not suffer from GvHD after the allogeneic hematopoietic cell transplantation.

### Donor Selection Methods of the Present Technology

In some embodiments, the patient requiring HCT expresses HLA-Bw4. Typically, for a patient requiring HCT, more than one donor is identified. Thus, for a given patient, potential donors, equivalent based on HLA-matching, would be screened in accordance with the methods and systems disclosed herein in order to select the best donor based on donor and recipient KIR3DL1 and HLA-Bw4 allele combinations.

In one aspect, the present disclosure provides methods and systems for selecting a donor for treating leukemia in a patient recipient. In another aspect, the present disclosure provides methods and systems for ranking candidate donors for treating leukemia in a patient recipient. In addition, in some aspects, the present disclosure provides a non-transitory computer readable storage medium having computer readable program for operating on a computer for performing the method of a) selecting a donor for treating leukemia in a patient recipient from one or more candidate donors or b) ranking candidate donors for treating leukemia in a patient recipient. In some embodiments, the methods and systems described herein can be performed by one or more processors. In some embodiments, the one or more processors can be configured to receive a patient profile of a patient recipient having a HLA-B genotype. In some embodiments, the patient recipient has leukemia. The one or more processors can be configured to select a type of donor based on the patient profile. The one or more processors can be configured to provide an output including a list of candidate donors that are suitable matches for the patient recipient based on the selected donor type. In some embodiments, the one or more processors can determine that the patient profile includes a HLA-B allele. The one or more processors can then select a donor type to identify candidate donors that also express the same HLA-B allele as the patient recipient. The one or more processors can, for each candidate donor, identify the KIR3DL1 allele expressed by the candidate donor and rank the candidate donors based on the type of KIR3DL1 allele expressed by the candidate donors. In some embodiments, the output can be displayed on a display screen. In some embodiments, the output can be used to select a donor.

In some embodiments, the list of candidate donors can be ranked according to a suitability of each candidate donor and the patient recipient. In some embodiments, each candidate donor can be assigned a score indicating compatibility with the patient recipient. In some embodiments, the score can be based on a candidate donor's expression of KIR3DL1-N and HLA-Bw4. In some embodiments, the HLA-Bw4 allele is HLA-Bw4-80T or HLA-Bw4-80I. Additionally or alternatively, in some embodiments, the candidate donor expresses KIR2DS1 and HLA-C1. In certain embodiments, the candidate donor expresses HLA-C1 or HLA-C2. In some embodiments, the one or more processors can rank candidate donors expressing KIR3DL1-N higher than candidate donors expressing KIR3DL1-L or KIR3DL-H. In some embodiments, the one or more processors can rank candidate donors expressing KIR3DL1-L higher than candidate donors expressing KIR3DL1-H responsive to determining that the patient recipient and candidate donor both express HLA-Bw4-80I. In some embodiments, the one or more processors can rank candidate donors expressing KIR3DL1-H higher than candidate donors expressing KIR3DL1-L responsive to determining that the patient recipient and candidate donor both express HLA-Bw4-80T.

### EXAMPLES

The present technology is further illustrated by the following Examples, which should not be construed as limiting in any way.

### Example 1: Experimental Materials and Methods

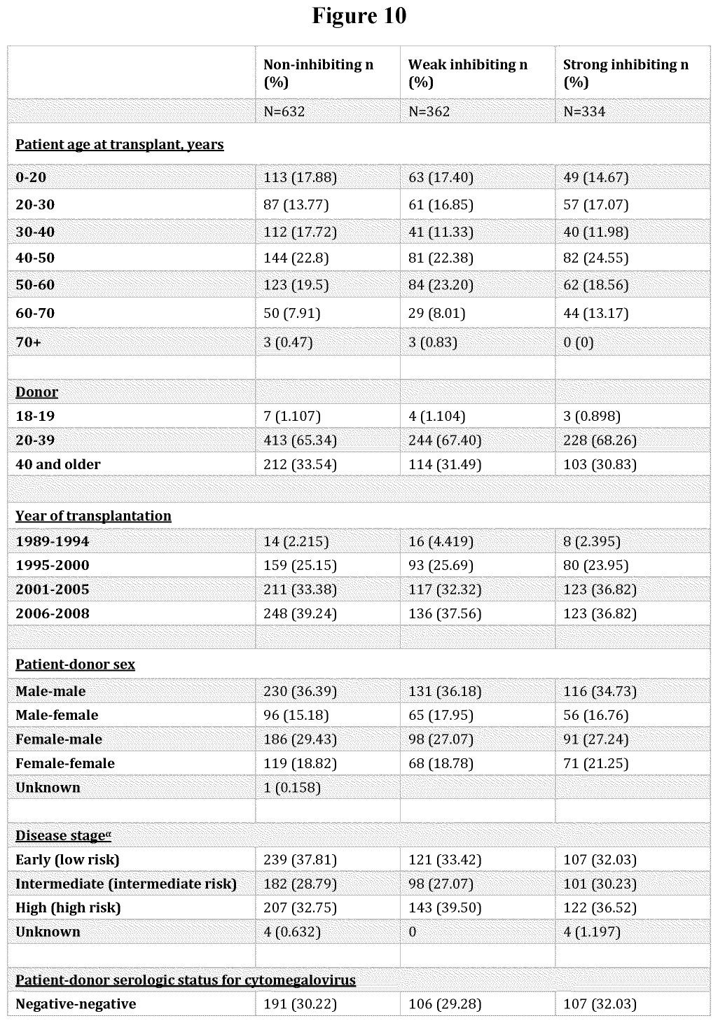

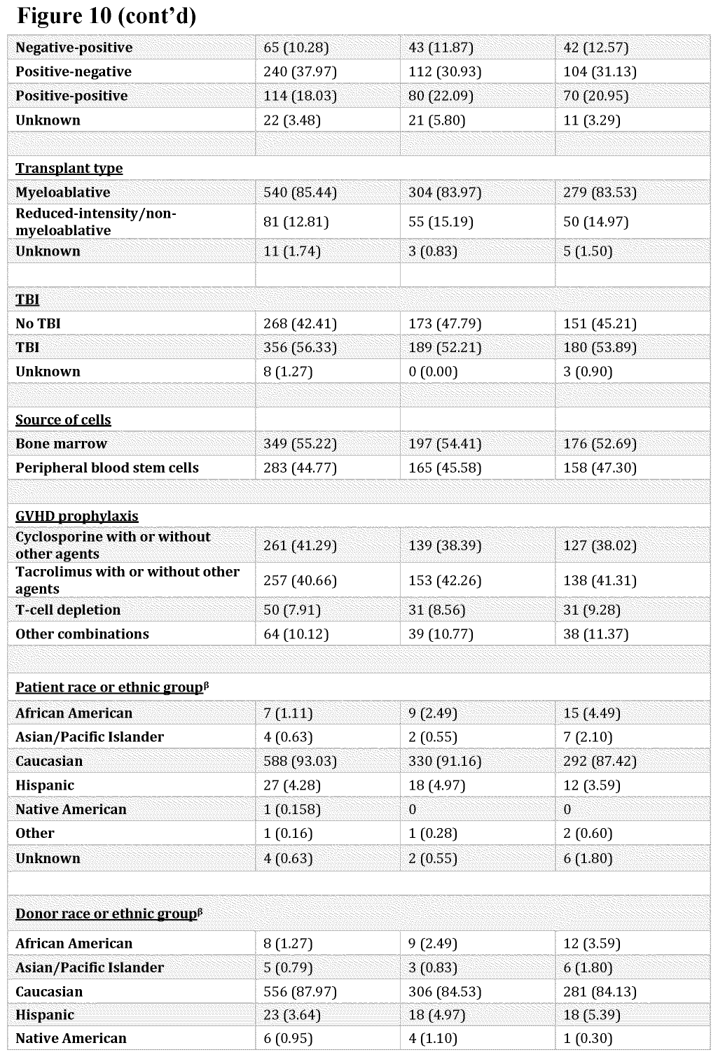

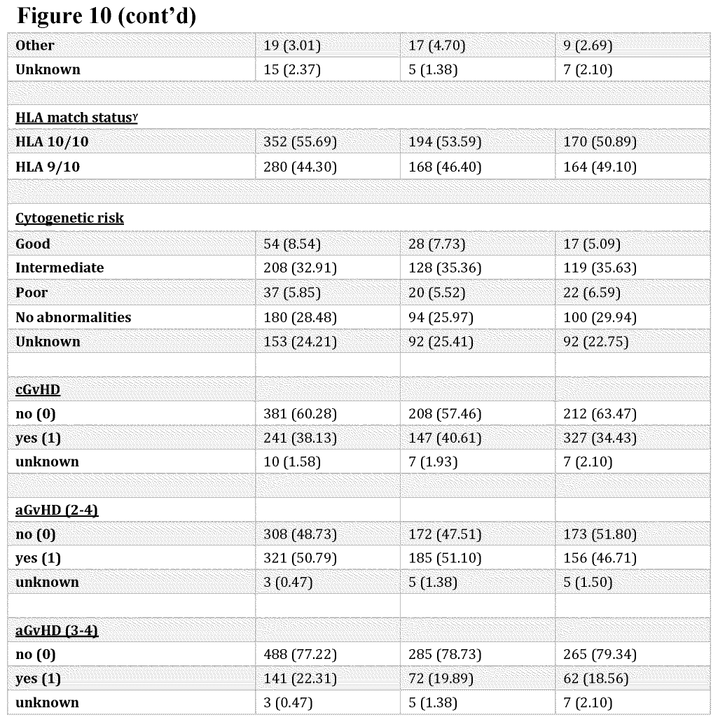

Clinical Samples and Healthy Donor PBMC. AML patients (n=1328) who received an allograft from a 9/10 or 10/10 HLA-matched unrelated donor were evaluated. The National Marrow Donor Program (NMDP) facilitated all transplants, and all donor-patient pairs for whom HLA typing and donor DNA were available were included in this study (FIG. 10). Clinical data, HLA genotyping, sequence-based typing for KIR3DL1 alleles, and genomic DNA were provided by the Center for International Blood and Marrow Transplant Research. Studies were performed in compliance with federal regulations pertaining to the protection of human research participants and approved by the NMDP IRB.

Patients and donors provided informed written consent for research. Healthy anonymous donor PBMC were collected from buffy coats obtained from the New York Blood Center (New York, N.Y.) (Boudreau J E et al., J Immunol 196:3398-3410 (2016)). Studies were approved by the Memorial Sloan Kettering Cancer Center (MSKCC) IRB.

Donor KIR and KIR3DL1 Typing. KIR genotyping was performed using sequence-specific PCR (Hsu K C et al., J Immunol 169:5118-5129 (2002), Vilches C et al., Tissue Antigens 70:415-422 (2007)) or KIR SSOP (Life Technologies, Grand Island, N.Y.; One Lambda, Canoga Park, Calif.). Sequence-based KIR3DL1 allele typing was available for 299 donors (Belle I et al., Tissue Antigens 71:434-439 (2008), Lebedeva T V et al., HIM 68:789-796 (2007), Levinson R D et al., Genes Immun 9:249-258 (2008)). Using multiplex PCR, 1029 donors were assessed for KIR3DL1 subtypes (Boudreau J E et al., PLoS ONE 9:e99543 (2014)). Allele frequencies were similar to previous findings.

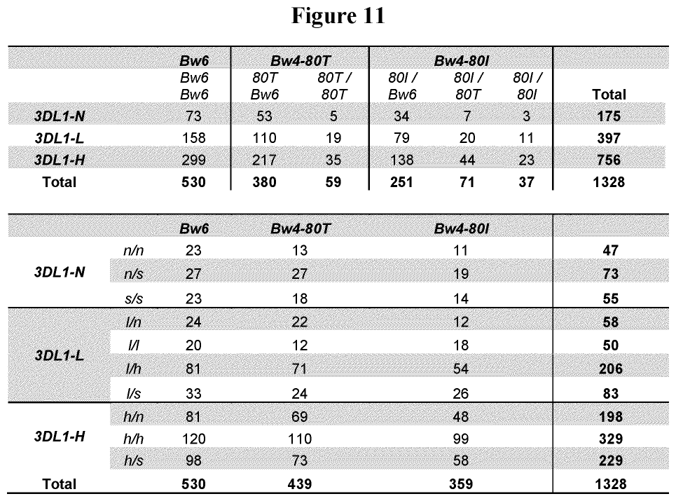

KIR3DL1 alleles were classified as KIR3DS1, KIR3DL1-high (h), KIR3DL1-low (l) or KIR3DL1-null (n) subtypes based on known polymorphisms and expression (FIGS. 10-12); Bw6, Bw4-80T and Bw4-80I epitopes were assigned using the ImmunoPolymorphism database (EMBL-EBI, Cambridgeshire UK). KIR3DL1 and HLA-B were grouped based on their compound subtypes (FIGS. 11-12).

AML Cell Lines and Primary Blasts. AML blasts were collected from patient peripheral blood and bone marrow. Cell lines were confirmed mycoplasma-negative, and HLA was determined by sequencing (Histogenetics, Ossining, N.Y.) or KIR ligand (Olerup, West Chester, Pa.) typing. Cells were maintained in RPMI-1640, supplemented with 10% FBS. To upregulate HLA expression, cells were cultured for 3 days with 1000 IU/mL human IFN-γ (Peprotech, Rocky Hill, N.J.).

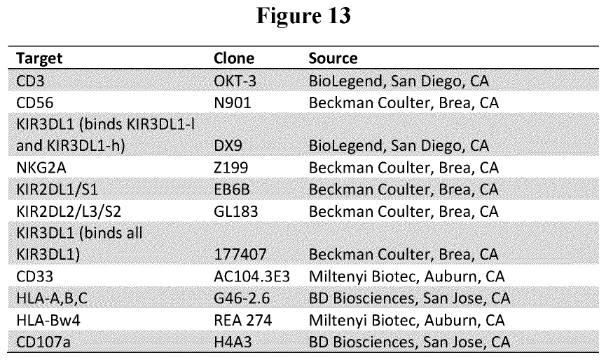

FACS and in vitro Cytotoxicity. NK and target cells were co-cultured (1:1) with anti-CD107a to quantify degranulation. Where specified, AML cell lines were pre-treated with 10μg/mL anti-HLA-B, -C antibody (4E, MSKCC Monoclonal Antibody and Bioresource core facility) to inhibit KIR engagement. Following 6 h co-culture, PBMC were stained for FACS using live/dead fixable stain (Life Technologies) and fluorochrome-tagged antibodies (FIG. 13).

To quantify cytotoxicity, target cells were stained using CFSE (Sigma, St. Louis, Mo.), co-cultured with PBMC (3:1 effector:target) for 48 h at 37° C., 5% CO₂ and counterstained with DAPI (Sigma). The anti-KIR3DL1/S1 antibody Z27 was included to block KIR3DL1/HLA-Bw4 interaction.

Statistical Analysis. All models used Cox regression for the time-to-event post-HCT outcomes for relapse and death. Probabilities of overall survival and relapse were obtained using Kaplan-Meier and cumulative incidence estimates, respectively, where death without relapse was regarded as a competing risk for relapse. Multivariate analysis adjusted for patient age, conditioning regimen, T cell depletion, graft type, disease status, CMV, gender match, and HLA-match. For functional studies, one-way ANOVA using Tukey's post-hoc test or Kruskal-Wallis non-parametric assessments with Dunn's correction were used. To assess KIR3DL1-n⁺ versus receptor-negative populations, paired Student's t-tests compared NK cells derived from the same donor. Clinical and functional analyses were completed in R and Prism 6 software, respectively, and p<0.05 was considered statistically significant.

### Example 2: HLA-Bw4 Subtypes Hierarchically Inhibit Primary NK Cells

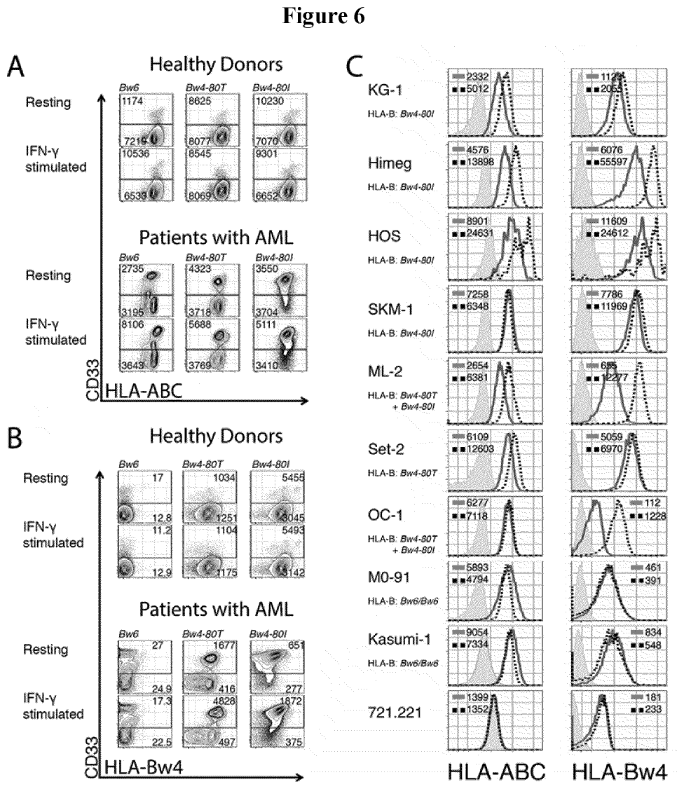

AML patients lacking HLA ligands for donor inhibitory KIR have lower relapse and higher survival following HCT compared to patients exhibiting all KIR ligands, suggesting that HLA expression on the tumor inhibits NK function in vivo. FIGS. 6(A)-6(C) demonstrate that total HLA, and specifically HLA-Bw4, is expressed on CD33⁺ AML cell blasts and cell lines. Treatment with IFN-γ, to mimic inflammation in HCT further upregulates HLA.

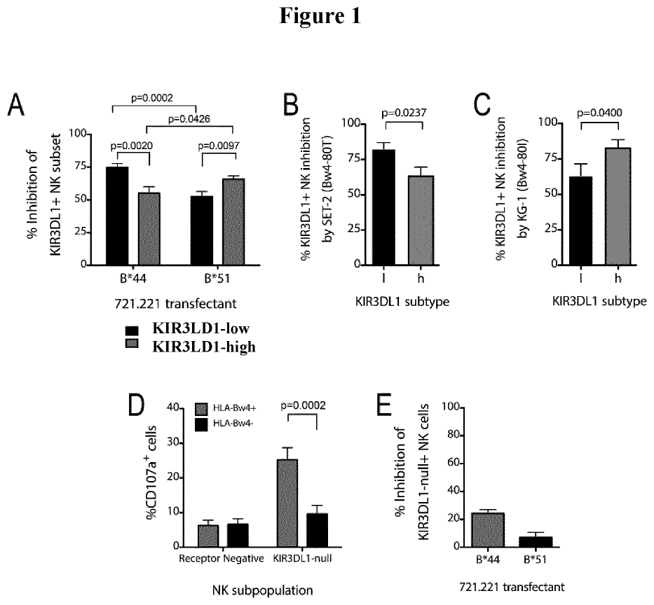

In HLA-matched HCT, educated NK cells are at risk for inhibition by HLA expressed on the tumor. To determine if NK cells with specific KIR3DL1 subtypes are variably inhibited by HLA-Bw4 subtypes, the inhibition of NK cells single positive (spNK) for KIR3DL1 by HLA-Bw4⁺ target cells was evaluated. To simulate the HLA-matched HCT setting, primary NK cells were challenged with high or low KIR3DL1 expression from Bw4-80T⁺ or Bw4-80I⁺ individuals with HLA-Bw4-matched targets. Among Bw4-80T donors, KIR3DL1-l⁺ spNK cells were more inhibited than KIR3DL1-h⁺ spNK by the 721.221 transfectant expressing the Bw4-80T allele HLA-B*44:02 and by the Bw4-80T⁺ AML cell line SET-2 (FIGS. 1(A)-1(B)). The opposite was observed among Bw4-80I donors: KIR3DL1-h⁺ spNK cells were more inhibited than KIR3DL1-l⁺ spNK cells by 721.221 target cells expressing the Bw4-80I allele HLA-B*51:01 and by the Bw4-80I⁺ AML cell line KG-1 (FIG. 1(A) and FIG. 1(C)). The higher inhibition of KIR3DL1-l⁺ NK cells by Bw4-80T relative to -80I was unexpected based on the relative binding affinity.

### Example 3: KIR3DL1-null⁺ NK Cells are Cytotoxic, Yet Insensitive to Inhibition

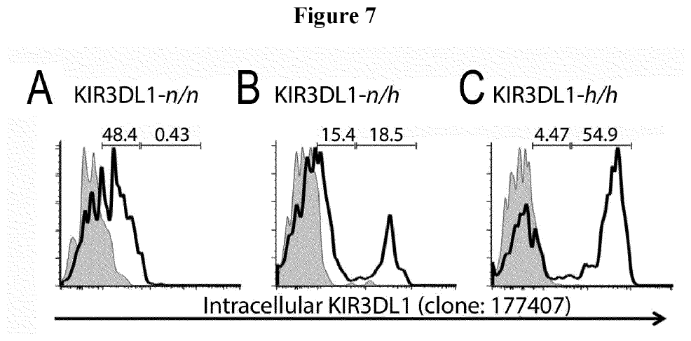

FIGS. 7(A)-7(C) demonstrate optimized staining for KIR3DL1 was optimized to determine if KIR3DL1-n would educate NK cells. KIR3DL1-n⁺ spNK cells from HLA-Bw4⁺ donors, but not HLA-Bw4-negative donors, were highly responsive to 721.221 targets (FIG. 1(D)), but insensitive to inhibition by HLA-Bw4⁺721.221 target cells (FIG. 1(E)), indicating that they are educated for effector response, while refractory to inhibition.

Accordingly, these results demonstrate that the methods of the present technology are useful for treating leukemia in a subject and/or protecting a leukemia patient from leukemic relapse following allogeneic hematopoietic cell transplantation.

### Example 4: KIR3DL1 and HLA-Bw4 Subtype Combinations Predict Differential Leukemotoxicity

To determine how AML killing was affected by differences in inhibition of KIR3DL1-expressing NK cells, PBMC from individuals representing different patient subgroups were co-incubated with Bw4 subtype-matched AML target cells. Diploid haplotypes were assigned into KIR3DL1 subgroups KIR3DL1-L or KIR3DL1-H (FIG. 11). A third subgroup represented donors exclusively exhibiting KIR3DL1-n and/or KIR3DS1 subtypes (KIR3DL1-N).

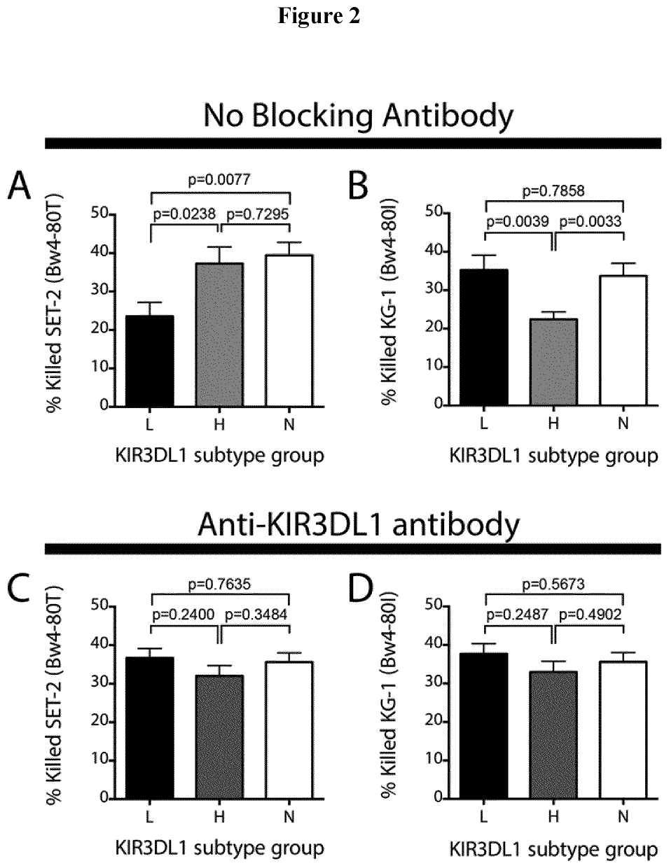

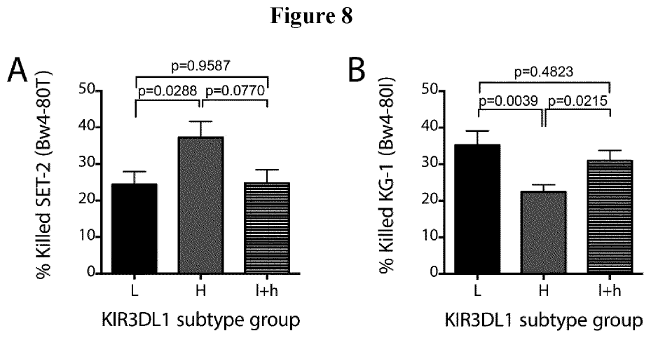

Among Bw4-80T individuals, KIR3DL1-H⁺ PBMC killed the Bw4-80I⁺ AML target more efficiently than KIR3DL1-L⁺ PBMC (FIG. 2(A)). In contrast, among Bw4-80I individuals, KIR3DL1-L⁺ PBMC killed Bw4-80I⁺ AML targets more efficiently than KIR3DL1-H⁺ PBMC (FIG. 2(B)). Antibody blockade of KIR3DL1 equalized target cell lysis between groups, indicating that the differences in cytotoxicity could be explicitly attributed to differential inhibition of the KIR3DL1⁺ cell population (FIG. 2(C)-2(D)). KIR3DL1-NPBMC demonstrated high cytotoxicity against both cell lines, unchanged by addition of anti-KIR3DL1/S1, reflecting simultaneous education and insensitivity to inhibition. NK cells heterozygous for KIR3DL1-l+h demonstrated greater killing of Bw4-80I⁺ than Bw4-80I⁺ targets (FIGS. 8(A)-8(B)).

### Example 5: Strong Inhibitory Subtypes of KIR3DL1 and HLA -B are Associated with Increased AML Relapse and Mortality

To determine if inhibitory sensitivities established by KIR3DL1 and HLA-Bw4 subtypes affect AML control, donor KIR3DL1 and HLA-B subtypes were retrospectively evaluated for 1328 AML patients receiving an unrelated HLA-compatible HCT. Neither donor-recipient HLA-B epitope, Bw4 subtype, nor KIR3DL1 subtype alone was associated with overall mortality or relapse. In contrast, any impact of KIR3DL1 on outcomes, particularly relapse, was dependent on the HLA-Bw4 subtype (test of interaction: p=0.06).

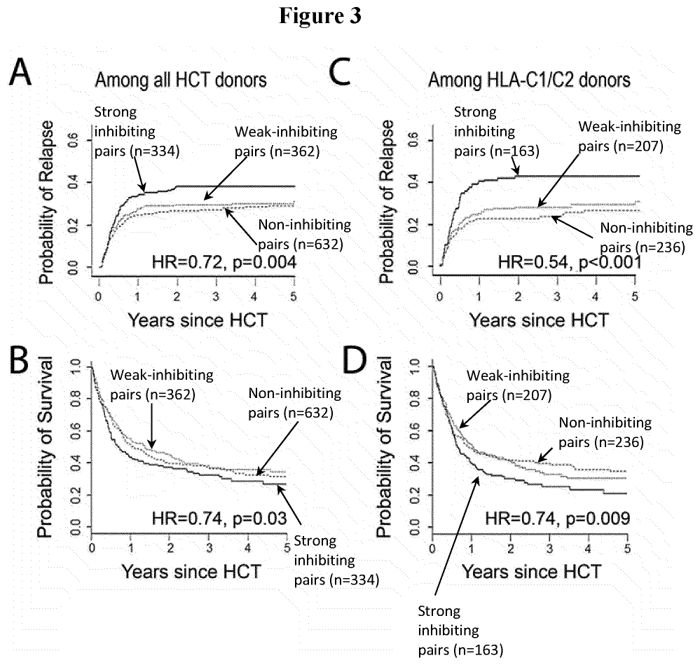

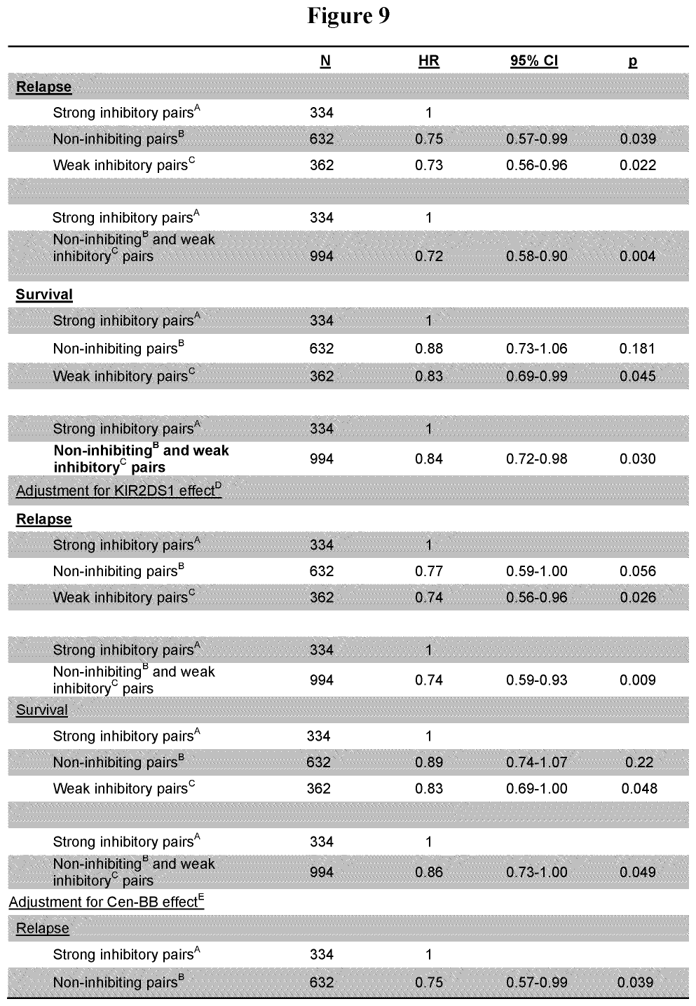

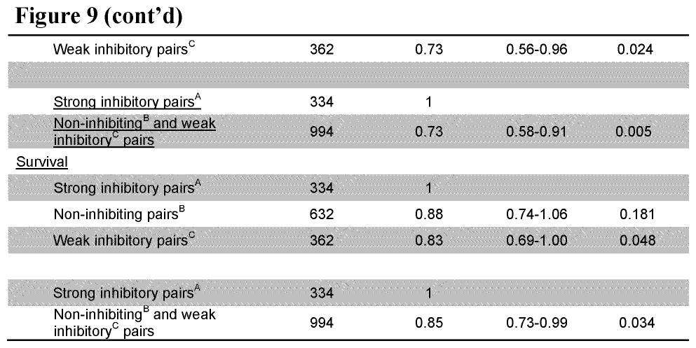

Based on the relative inhibitory and cytotoxic strengths against targets in vitro, KIR3DL1-H+Bw4-80I and KIR3DL1-L+Bw4-80T were considered collectively as strong inhibiting pairs; the reciprocal combinations were considered as weak inhibiting pairs. A third “non-inhibiting” classification was comprised of donors with Bw6 and/or KIR3DL1-N. In a multivariate analysis, weak inhibiting pairs demonstrated significantly lower relapse (HR=0.73, p=0.019) and mortality (HR=0.83, p=0.041) compared with strong inhibiting pairs; non-inhibiting pairs were similarly beneficial (relapse, HR=0.72, p=0.008; mortality, HR=0.87, p=0.064 (FIG. 9). Combined, donors with weak or non-inhibiting pairs were associated with superior outcomes compared with strong inhibiting pairs (relapse: HR=0.72, p=0.004, FIG. 3(A); mortality: HR=0.84, p=0.03, FIG. 3(B)).

Removal of the 55 KIR3DS1 homozygous donors did not alter our conclusions; therefore, the benefit of non-inhibiting pairs was not due to enrichment for KIR3DS1 or other activating receptors in positive linkage disequilibrium (FIG. 9). There was no impact of Bw4 epitopes encoded by HLA-A alleles. The KIR2DS2 and KIR2DL2 genes are located on the centromeric B region, cenB. Correcting for cenBB did not alter KIR3DL1/Bw4 effects (FIG. 9).

### Example 6: Weak/Non-Inhibiting KIR3DL1/HLA-B Subtype Combinations are Most Protective in HLA-C1/C2 HCT

Among HCT recipients, 40% exhibit HLA-Bw4/C1/C2, or “all KIR ligands,” a configuration associated with higher relapse and mortality compared with patients lacking at least one KIR ligand. Segregating HCT pairs according to HLA-C KIR ligands, the data demonstrate that the protective effects of weak/non-inhibiting vs strong inhibiting combinations for relapse (HR 0.54, p<0.001) and mortality (HR 0.74, p=0.009) were most evident in the “high risk” HLA-C1/C2 transplant pairs (FIG. 3(C)-3(D)).

Accordingly, these results demonstrate that the methods of the present technology are useful for treating leukemia in a subject and/or protecting a leukemia patient from leukemic relapse following allogeneic hematopoietic cell transplantation.

### Example 7: Benefits of KIR2DS1 and KIR3DL1/HLA-B are Distinct

In the present cohort of 1328 donor-patient pairs, a benefit of donor KIR2DS1+HLA-C1 was demonstrated (relapse: HR=0.79, p=0.04; mortality: HR=0 . 89, p=0.12). The protection associated with weak/non-inhibiting KIR3DL1/HLA-B subtype combinations was not altered by correction for KIR2DS1/HLA-C1 (FIG. 9).

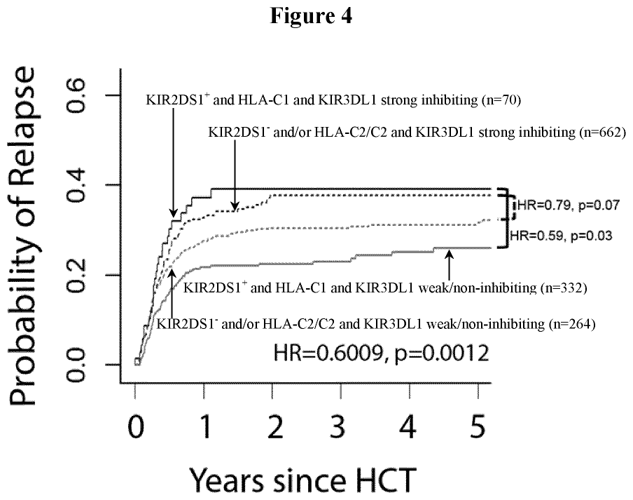

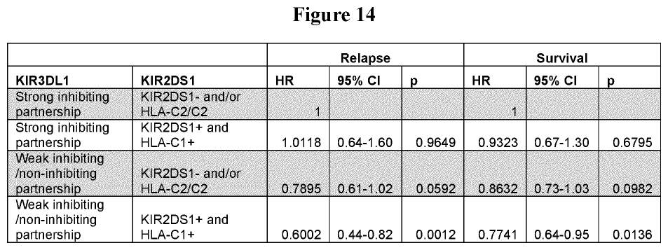

Donors exhibiting the combined benefits of weak/non-inhibiting KIR3DL1/HLA-B with KIR2DS1/HLA-C1 conveyed the lowest relapse and highest survival to patients. Strong inhibiting KIR3DL1/HLA-Bw4 partnerships exhibited the highest relapse and mortality, which could not be improved by combination with KIR2DS1+HLA-C1 (FIG. 4, FIG. 14). Therefore, while the benefits of KIR3DL1/HLA-B and KIR2DS1/HLA-C1 are separate, the former exhibits primacy in HCT outcomes.

Accordingly, these results demonstrate that the methods of the present technology are useful for treating leukemia in a subject and/or protecting a leukemia patient from leukemic relapse following allogeneic hematopoietic cell transplantation.

### Example 8: KIR3DL1 Subtypes: A Novel Donor Selection Criterion

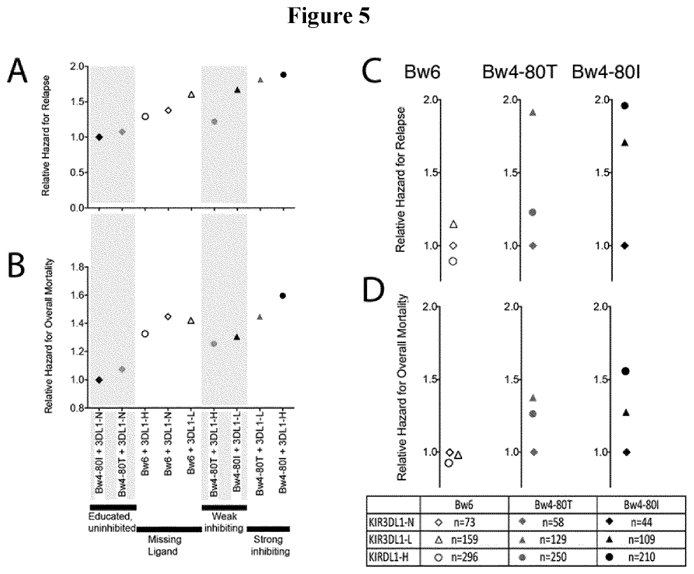

To examine the association of increased NK inhibition with risk of failure, relative hazards were determined for AML relapse and survival among all KIR3DL1/HLA-B subtype combinations in HLA-matched HCT, clustering groups based on in vitro assessments of education and inhibitory sensitivity (FIGS. 5(A)-5(B)). Bw6 donor-patient pairs experienced intermediate protection, reflecting the “missing ligand” benefit, but the lowest relapse and mortality was observed among KIR3DL1-N+HLA-Bw4, where cells were educated but refractory to inhibition. KIR3DL1-Bw4 partnerships predictive of weak inhibition (Bw4-80T+KIR3DL1-H or Bw4-80I+KIR3DL1-L) were associated with intermediate relapse and mortality, demonstrating a benefit of education and a weak sensitivity to inhibition. The strong-inhibiting partnerships (Bw4-80I+KIR3DL1-H and Bw4-80T+KIR3DL1-L) were associated with the highest relapse and mortality, demonstrating that a strong inhibitory signal overrides the benefit of NK education.

Outcomes among HLA-B patients groups were compared to understand whether donor selection based on KIR3DL1 subtypes may be an effective intervention to improve AML control (FIGS. 5(C)-5(D)). There were no distinct advantages among donor KIR3DL1 subtypes in Bw6⁺ donor-recipient pairs. For Bw4-80T⁺ recipients, the greatest protection from relapse occurred if donors exhibited KIR3DL1-H (HR=0.65, p=0.031) or KIR3DL1-N (HR=0.52, p=0.058) compared to donors with KIR3DL1-L; overall mortality followed the same trend. For Bw4-80I⁺ recipients, KIR3DL1-N donors were most protective for relapse (HR=0.52, p=0.055) and mortality (HR=0.64, p=0.054) compared to KIR3DL1-H donors.

Accordingly, these results demonstrate that the methods of the present technology are useful for treating leukemia in a subject and/or protecting a leukemia patient from leukemic relapse following allogeneic hematopoietic cell transplantation.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for treating leukemia in a subject in need thereof, comprising administering to the subject an effective amount of hematopoietic cells obtained from a candidate donor, wherein the candidate donor has a KIR3DL1-HLA-B allele combination of KIR3DL1-N and HLA-Bw4.

2. The method of claim 1, wherein HLA-Bw4 is HLA-Bw4-80T or HLA-Bw4-80I.

3. The method of claim 1, wherein the candidate donor expresses KIR2DS1 and HLA-C1.

4. The method of claim 1, wherein the candidate donor expresses HLA-C1 or HLA-C2.

5. The method of claim 1, wherein the leukemia is a chronic leukemia or an acute leukemia.

6. The method of claim 1, wherein the leukemia is acute myelogenous leukemia (AML), chronic myelogenous leukemia (CIVIL), chronic lymphocytic leukemia (CLL), acute lymphocytic leukemia (ALL), hairy cell leukemia, T cell prolymphcytic leukemia (T-PLL), or large granular lymphocytic leukemia.

7. The method of claim 1, wherein the hematopoietic cells obtained from the candidate donor comprise hematopoietic stem cells.

8. The method of claim 1, wherein the hematopoietic cells obtained from the candidate donor are derived from bone marrow, peripheral blood cells, or umbilical cord.

9. The method of claim 1, further comprising treating the subject with chemotherapy and/or radiation.

10. The method of claim 1, wherein the subject is human.

11. A method for protecting a leukemia patient from leukemic relapse following allogeneic hematopoietic cell transplantation (HCT), comprising administering to the leukemia patient an allogeneic hematopoietic graft derived from a candidate donor, wherein the candidate donor has a KIR3DL1-HLA-B allele combination of KIR3DL1-N and HLA-Bw4.

12. The method of claim 11, wherein HLA-Bw4 is HLA-Bw4-80T or HLA-Bw4-80I.

13. The method of claim 11, wherein the candidate donor expresses KIR2DS1 and HLA-C1.

14. The method of claim 11, wherein the candidate donor expresses HLA-C1 or HLA-C2.

15. The method of 14 claim 11, wherein the leukemia is a chronic leukemia or an acute leukemia.

16. The method of 15 claim 11, wherein the leukemia is acute myelogenous leukemia (AML), chronic myelogenous leukemia (CIVIL), chronic lymphocytic leukemia (CLL), acute lymphocytic leukemia (ALL), hairy cell leukemia, T cell prolymphcytic leukemia (T-PLL), or large granular lymphocytic leukemia.

17. The method of 16 claim 11, wherein the leukemia patient is at the early, intermediate or advanced stage of leukemia.

18. The method of 17 claim 11, wherein the allogeneic hematopoietic graft is a bone marrow graft or a peripheral bone stem cell graft.

19. The method of 18 claim 11, wherein the allogeneic hematopoietic cell transplantation is ablative, T-cell depleted, or T-cell replete.

20. The method of 19 claim 11, wherein the leukemia patient does not suffer from GvHD after the allogeneic hematopoietic cell transplantation.
